(19) United States
(12) Patent Application Publication
(10) Pub. No.: US 2005/0065600 A1
(43) Pub. Date: Mar. 24, 2005

(54) Prosthetic heart valve of pyrolytic carbon

(76) Inventor: Harischandra K. Mhatre, Mumbai (Bombay) (IN)

Correspondence Address: OLSON & HIERL, LTD.;36th Floor, 20 North Wacker Drive, Chicago, IL, 60606, US

(21) Appl. No.: 10/666,457

(22) Filed: Sep. 19, 2003

(51) Int. Cl. A61F 2/24

(52) U.S. Cl. 623/2.31; 623/2.38; 29/890.126

(57) ABSTRACT

A prosthetic heart valve is provided that includes a ring structure that incorporates integrally a bearing block, a separate bearing block having two pins for accurate location that seats in a window in the ring structure and that is held in place by a circlip, and a pair of leaflets that are disposed in the passageway of the ring structure. The valve utilizes pyrolytic carbon in the ring structure, separate bearing block and leaflets. The valve has a simple structure, is durable and reliable, and is assembled without flexure or distortion of the ring structure, separate bearing block or leaflets.

### FIELD OF THE INVENTION

This invention relates to prosthetic heart valves, more particularly to prosthetic heart valves made with pyrolytic carbon.

### BACKGROUND OF THE INVENTION

The main function of a prosthetic heart valve is to provide unidirectional, uniform and reliable flow of blood in the human circulatory system. Additionally, such a valve should be comprised of materials that are compatible with body tissues when implanted and should be capable of long duty life.

Prosthetic heart valves having two leaflets, also identified as bi-leaflet or double leaflet heart valve prostheses, are known in the prior art. Such a structure typically incorporates an annular member, also identified as a ring member, and two cooperating leaflets that are intended to open and close the passageway through the ring member responsive to blood flow.

Because pyrolytic carbon appears to be compatible with human body tissues and to be suitable for implantation for extended time periods, and also to have desirable characteristics and features, such as great hardness, low friction, high durability, wear resistance, and deterioration resistance, it would perhaps be useful in prosthetic heart valves. Pyrolytic carbon, methods for coating substrate bodies with pyrolytic carbon, and methods for fabricating pyrolytic carbon structures are well known (see, for example, Bokros et al. U.S. Pat. Nos. 3,298,921; 3,399,969; 3,526,005 and 3,547,676).

However, attempts to employ pyrolytic carbon in bi-leaflet prosthetic heart valves have presented problems. For example, assembly of preformed and pyrolytic carbon coated leaflets with a preformed and pyrolytic carbon coated ring structure was not successful because of the characteristically rigid and relatively inflexible nature of pyrolytic carbon. The engagement of the ears of the leaflets with recesses defined in the ring member involved flexing which injured the pyrolytic carbon coating and deleteriously affected the subsequent use life and usability of the prosthesis especially between adjacent bearing surfaces even when only minimal contact occurred therebetween.

A little component flexing during prosthesis assembly is sometimes achievable either by providing two arm-like projections on the ring member that are preferably flat and by using leaflets that are adapted for engaging recesses provided on either the blood inlet or the outlet side of the ring member. The projections need to be thin in cross-section so as to make them slightly more flexible than otherwise. However, the undesirable possibilities of producing residual permanent deformation of the ring member or of cracking the ring member during the prosthesis assembly process cannot be completely eliminated. Also, with such thin structural members, the resulting minimum bearing engagement between adjacent surfaces makes the ears of each leaflet prone to slipping or dislodgement from their associated recesses in the resulting prosthesis after implantation and during use. Further, using flat, arm-like projections on the ring member can make the ring member very cumbersome, risky to handle and utilize, and can require careful extra efforts to insert the resulting heart valve prosthesis into an appropriate position in the patient's heart, particularly during an aortic valve replacement operation.

The alternative of a thin cross-section for the ring member hardly provides the required minimum rigidity for a ring member. If such a ring member is used, the resulting heart valve prosthesis is an intrinsically weak device. The unitary construction of the ring member with its bearing surface defining recesses to accommodate the ears of the leaflets results in an inherent inability to adjust axial play precisely in the hinging mechanism, such as is necessary to minimize malfunction, disruption and consequent dislodgement of the leaflets over a period of time in use.

Thus, the provision of providing either flat arm projections on the ring member, or a thin cross-section in the ring member, together with achieving the minimum bearing surface engagement needed between ears and recesses, and the inability to adjust or accurately control axial end-play in the hinging mechanism, limits long term reliable service of the presently available prior art bi-leaflet heart valve prostheses.

Since safety of human life is involved, improved structure and fabrication processes are needed to achieve efficient and reliable service of bi-leaflet heart valve prostheses fabricated with pyrolytic carbon. Even a very low failure rate for heart valve prostheses is undesirable.

Moreover, an improved pyrolytic carbon-containing, bi-leaflet heart valve prosthesis should achieve optimal expected long service life characteristics.

There is a need for a new and improved heart valve prosthesis of the bi-leaflet type which utilizes pyrolytic carbon and which surpasses the performance and improves the reliability of prior art heart valve prostheses. The present invention provides such an improved heart valve prosthesis.

### SUMMARY OF THE INVENTION

The present invention relates to an improved prosthetic heart valve of the bi-leaflet type which incorporates pyrolytic carbon and which achieves improved reliability and durability, easy assembly, long use life, and also improved ease and reduction of cost in manufacture and assembly.

The inventive heart valve prosthesis incorporates an annular or ring-like structure in which a bearing block receiving window is provided. In the window, a bearing block is demountably received and reliably retained by means of a preferably cross-sectionally circular circlip that associates with groove means in the annular structure. The bearing block has a taper about its perimeter which cooperatingly associates with a corresponding mating taper defined about the periphery of the window, and preferably these components are generally rectangularly configured. An integrally formed portion of the annular structure that is located diametrically opposite the receiving window and the associated bearing block is adapted to function as the needed second bearing block. These respective bearing blocks each have two recesses defined therein.

The leaflets of the prosthesis are disposed in and across the passageway in the annular structure, and each leaflet has a pair of projecting ears that in the assembled prosthesis each cooperate and associate with a different recess defined in the combination of annular structure with associated separate bearing block, thereby to receive and support the ears so that the leaflets are adapted for achieving precise pivotable movements in the passageway to provide valve-defining operation without the possibility of leaflet slippage.

During prosthesis assembly, and the associating of the leaflets with the annular structure and the separate bearing block, the separate bearing block is loosened relative to the annular structure. Assembly is achieved without flexure or bending of components (except for the circlip).

Except for the circlip, and optional but preferable locating pins employed for precisely positioning the separate block in the block receiving window, the components are comprised of pyrolytic carbon. The retaining circlip for the separate bearing block relative to the annular structure is comprised of a rust-proof spring steel and the locating pins are preferably comprised of rust-proof steel.

The product prosthesis is comprised of a minimum number of components and is a relatively simple structure of great durability that is human body compatible when implanted.

The prosthesis components can be separately and accurately fabricated with conventional processing including use of machine tools.

Assembly of the components is simple and reliable. No flexing, bending, or the like of components utilizing pyrolytic carbon is involved.

The product prosthesis is very reliable, provides excellent service for an extended time period, and is very efficient.

The pair of pivotable leaflets employed in the prosthesis combination functions to achieve a one-way valve and to control unidirectional blood flow through the passageway of the annular member. In the valve closed position, the edge portions of each leaflets are preferably configured to abut and engage sealingly with and against adjacent surface portions contacted therewith. Each leaflet is preferably flattened and its perimeter includes an arcuately extending outside edge region, a straight inside edge region, and a pair of flattened, ear-like projections, each one of which is located between a different pair of the adjacent opposite sides that extend between each end of the arcuate edge region and of the straight edge region.

Out-turned flanges at opposite ends of the annular member provide rigidity and strength. The bearing recesses achieved in each of the bearing blocks can be precisely located and sized.

Small, uniform clearances between respective adjacent portions of the leaflet ears and the bearing recesses are achieved by the precise interrelationship between components, such as that between the annular structure and the associated bearing blocks. Free, smooth, self-aligning spherical bearing surfaces are achieved for pivotal hinging-type movements of the leaflets relative to the bearing blocks. End play is adjusted by selective assembly and by precise construction, as those skilled in the art will readily appreciate.

The leaflet ears engage their associated bearing recesses to a desired, predetermined depth. Each of the leaflets is held securely while achieving a minute gap between each leaflet's ears and its associated bearing housing so that there is no possibility of malfunction.

The provision of pyrolytic carbon particularly in the regions of the bearing surfaces between the respective ears and the associated recesses, and the configuration of the bearing surfaces thus provided, ensures that the leaflet ears do not disengage or slip from bearings in the assembled prosthesis. The pyrolytic carbon in such regions provides a polished and hard surface.

Other and further features, purposes, objects, aims, advantages, embodiments and the like will be apparent to those skilled in the art from the present description taken with the appended drawings and the following claims.

### BRIEF DESCRIPTION OF THE DRAWINGS

In the drawings:

FIG. 1 is an isometric view of one embodiment of the inventive prosthetic bi-leaflet heart valve showing each leaflet in closed position;

FIG. 2 is a plan view of the valve of FIG. 1, with some parts thereof being shown in transverse section, this view being taken along the line II-II of FIG. 1;

FIG. 2A is a fragmentary, enlarged view showing a region of the perimeter edge portion of the bearing block receiving window that is defined in the medial side wall portion of the annular ring-like structure, portions thereof being broken away and portions thereof being shown in section, this view being taken from an interior region of the ring-like structure looking outwardly at the window perimeter edge region;

FIG. 3 is an isometric view similar to FIG. 1 but showing the valve in an exploded format;

FIG. 4 is a longitudinal sectional view taken along the axis of the FIG. 1 valve as indicated by the line IV-IV in FIG. 1, this view being taken through the pivot axes of the leaflets;

FIG. 5 is a longitudinal sectional view through the ring structure taken along the line V-V of FIG. 2;

FIG. 5A is a fragmentary longitudinal sectional view taken along the line VA-VA of FIG. 2;

FIG. 6 is a plan view of the circlip employed in the valve of FIG. 1;

FIG. 7 is a plan view of the inside face of the bearing block employed in the valve of FIG. 1 showing the bearing recesses defined therein;

FIG. 8 is a longitudinal vertical sectional view through the FIG. 7 bearing block taken along the line VIII-VIII of FIG. 7;

FIG. 9 is a fragmentary vertical sectional view through the FIG. 7 bearing block taken along the line IX-IX of FIG. 7;

FIG. 10 is a plan view of the upper face of the right-hand leaflet employed in the valve of FIGS. 1-4;

FIG. 11 is a fragmentary vertical sectional view through the FIG. 10 leaflet taken along the line XI-XI of FIG. 10;

FIG. 12 is a fragmentary enlarged detail view similar to a portion of FIG. 4 illustrating pivoting movements of the FIG. 10 leaflet relative of the separate bearing block in the valve of FIG. 1 with one position of the leaflet being shown in phantom;

FIG. 12A is a view similar to FIG. 12 but rotated 180° to illustrate the interior face of the integral bearing block and pivoting movements of the FIG. 10 leaflet relative to the integral bearing block;

FIG. 13 is a fragmentary, enlarged, detail, transverse sectional view through the region of the bearing block in the valve of FIG. 1 taken along the line XIII-XIII of FIG. 4;

FIG. 14 is a fragmentary, enlarged, detail, exterior side elevational view at the region of the bearing block in the valve of FIG. 1;

FIG. 15 is a view similar to FIG. 14 but showing an alternative embodiment that employs two circlips for retaining the bearing block in association with the ring structure; and

FIG. 16 is a fragmentary, enlarged, detail, transverse sectional view through the region of the bearing block of the alternative embodiment of FIG. 15.

### DESCRIPTION OF THE PREFERRED EMBODIMENTS

The inventive heart valve prosthesis is described and illustrated with reference to a particular now preferred embodiment 21 as shown in FIGS. 1 through 14, respectively. The prosthesis embodiment 21 includes in combination as cooperating components an annular ring-like structure 30, two valve leaflets 31 and 32, a bearing block 33, and a retaining ring or circlip 34. Except for the circlip 34, which is comprised of a non-rusting, resilient, spring steel, such as stainless steel or the like, and the optional but preferred locating pins 23, 24 which are preferably comprised of a non-rusting steel, all the components of the prosthesis 21 are comprised of pyrolytic carbon.

The pyrolytic carbon components are characterized by hard, highly polished, smooth, and glossy surface portions and capacity for long wear and resistance to wear with minimal frictional losses.

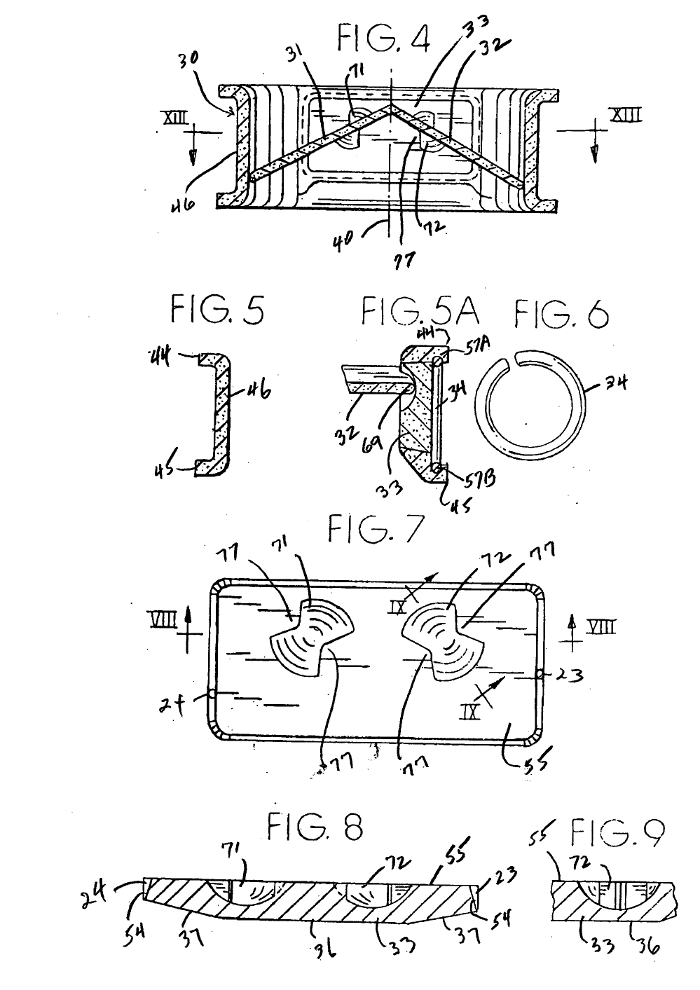

The ring structure 30 has a longitudinal axis 40 (shown, for example, in FIG. 4) and is generally annularly configured. Also, ring structure 30 preferably is somewhat axially elongated (relative to axis 40), with medial generally cylindrical sidewall portions 46 and with opposite end regions 41 and 42 that extend circumferentially in generally spaced, parallel relationship relative to each other. A central passageway 43 that is generally cross-sectionally cylindrical is defined through the ring 30 about the axis 40. Except for the bearing blocks 47, 33, exteriorly, the ring structure 30 is generally cross-sectionally cylindrical. The opposite end regions 41 and 42 are each provided with an integral, outwardly extending, terminal rim flange 44 and 45, respectively.

One location along the medial sidewall portions 46 is radially selectively thickened to provide an integrally formed bearing block region 47. The bearing block region 47 has a flat interior face 48 that extends chord-like (when the ring 30 is viewed axially) across the bearing block region 47 and parallel to the longitudinal axis 40. Relative to the face 48, the integral bearing block region 47 preferably has a generally rectangular perimeter configuration.

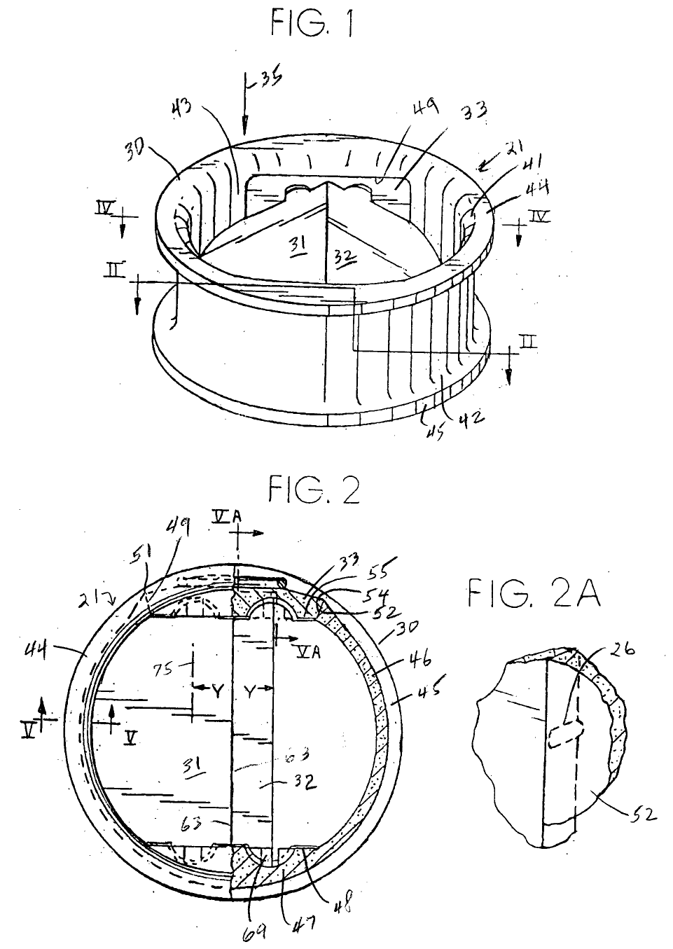

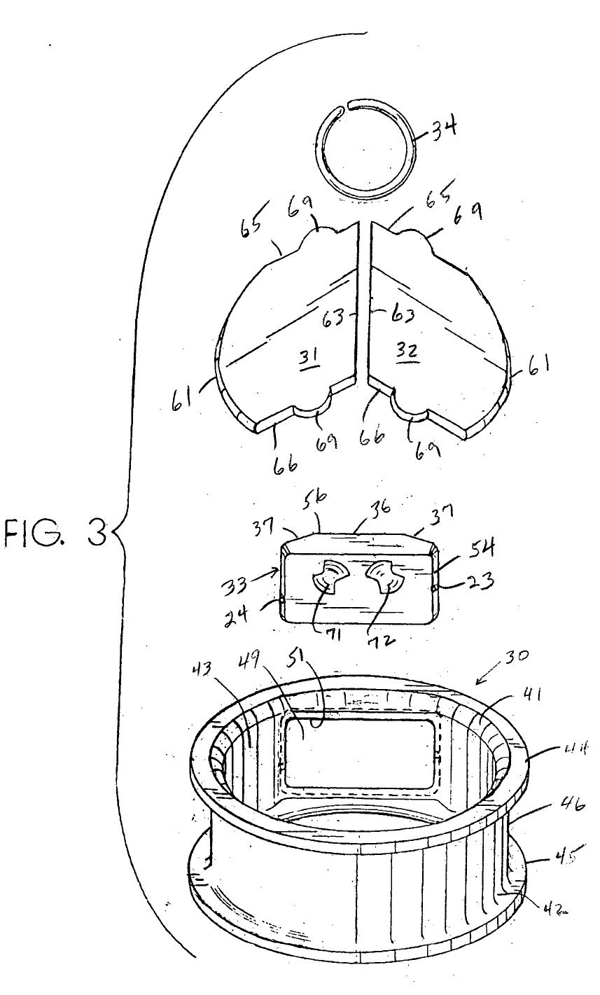

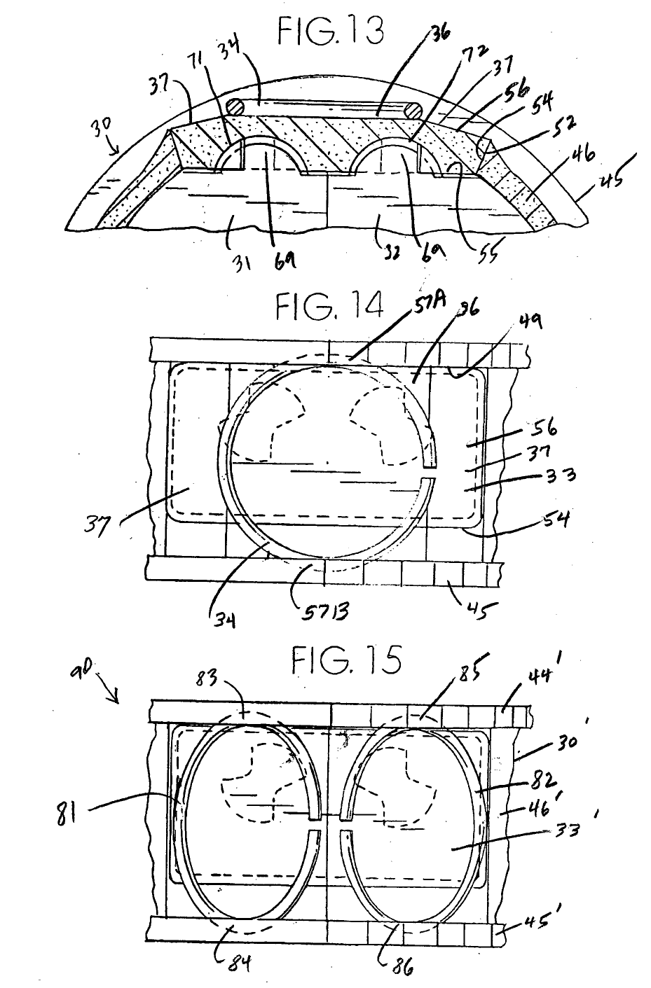

A second location along the sidewall portions 46 that is generally diametrically opposed to such one location (and to the integral bearing block region 47) has defined therethrough a bearing block receiving window 49 (see, for example, FIG. 3). Perimeter edge portions generally designated 51 of the window 49 are preferably radially thickened and include edge surface portions 52 (see, for example, FIG. 2 or FIG. 13) that are radially inclined (or beveled) and flattened to define a taper of the Morse-type or the like. The window 49 preferably has a generally rectangular perimeter configuration.

A separate, or independent, bearing block 33 is provided that has perimeter edge surface portions 54 that are inclined (or beveled) and flattened to defined a peripheral taper of the Morse-type or the like. The beveled perimeter edge surface portions 54 of block 33 are configured to matingly engage with and seat against the correspondingly matingly beveled edge surface portions 52 of the window 49 when the block 33 is initially suitably oriented and aligned relative to the ring 30 with the block 33 being initially (that is, before insertion into the window 49) spatially oriented and positioned at an adjacent exterior location relative to the ring 30. From such a location, the block 33 is radially (relative to the ring 30) moved into engagement with the window 49. The block 33 preferably has a rectangular perimeter configuration. Alternative geometric perimeter configurations may be employed if desired for the respective perimeter portions 54 and 51/52.

The block 33 has a flat interior face 55 that extends chord-like (when, in the assembled prosthesis 21, the ring 30 is viewed axially) across the bearing block 33. The exterior face 56 of the block 33 can be variously configured, but preferably has a flattened mid-region 36 that extends parallel to the interior face 55. Opposite side regions 37 on either side of mid-region 36 of exterior face 56 of block 33 are preferably each flattened and oriented symmetrically relative to each other and preferably each side region 37 terminates near to the adjacent exterior surface portions of the medial side wall portions 46 of the ring 30. When the block 33 is fully seated in the window 49, the interior face 55 extends parallel to the longitudinal axis 40, and the faces 55 and 48 are in spaced, parallel relationship relative to each other.

Preferably, as an aid to positioning a block 33 relative to a window 49, each opposite side of edge portion 54 of the block 33 is provided with a locating pin 23, 24 (see, for example, FIG. 3). Each pin 23, 24 is somewhat offset longitudinally relative to the other pin 23, 24 in a block 33 so as to enhance the specificity of the locating of the block 33 relative to the window 49. Correspondingly, each opposite side region of the edge surface portions 52 of window 49 has defined therein a locating pin receiving pocket 26 (see, for example, FIG. 2A), each pocket 26 being somewhat offset longitudinally relative to the other pocket 26 in a window 49. The respective locations of the pockets 26 are such that the pockets 26 are each positioned and configured to receive a different respective pin 23, 24 when a block 33 is seated in a window 49. Preferably, each pin 23, 24 is comprised of non-rusting steel that is, during fabrication, inset into its respective location in an edge surface portion 54 of block 33.

For purposes of mounting the block 33 in position in the window 49, the edge surface portions 51 of the window 49 in ring 30 have defined therein a perimetrically extending groove means that is achieved by a groove 57A that extends parallel to the rim flange 44 and by another groove 57B that extends parallel to the rim flange 45 (see, for example, FIG. 14). Various arrangements and configurations can be utilized for the groove means. In general, the groove means is preferably located so as to be adjacent to outer edge portions of the exterior face 56 of the separate bearing block 33 when the block 33 is seated, as above indicated, in the window 48. With the block 33 seated in the window 49, the circlip 34 is positioned so that portions thereof engage with the groove means, here grooves 57A and 57B, while other portions thereof contact the portions of the mid-region 36 of the exterior face 56, thereby to retain the block 33 seated in the window 49, as illustrated in FIG. 14, for example. The circlip 34 is preferably circular in plan view and is preferably circular in cross-section.

Various circlip configurations and various combinations of circlips and grooves can be employed, if desired. For example, an alternative heart valve prosthesis 90 incorporates an arrangement for groove means, circlip, and block as fragmentarily illustrated in FIGS. 15 and 16. In prosthesis 90, components corresponding to those in prosthesis 21 are similarly numbered but have prime marks added thereto for convenient identification purposes. Here, two circlips 81 and 82 are utilized, each one being retained by a different pair of grooves 83, 84 and 85, 86 respectively, to position and hold a block 33′. The block 33′ has a pair of flat faces 37′ that meet medially, and each face 37′ is in contacting relationship with a different circlip 81, 82 in the assembled prosthesis 21.

The leaflets 31 and 32 of prosthesis embodiment 21 are each configured so as to be complementary relative to each other so that the leaflets 31 and 32 can be cooperatively disposed across the passageway 43. The leaflets 31 and 32 can be considered to generally be mirror images of each other, and each has a generally flattened main body portion, and a perimeter that includes an outer rounded edge portion 61, an inner straight edge portion 63, and a pair of transversely spaced, generally parallel, straight, medial edge portions 65, 66 that interconnect between the opposite end regions of the outer rounded portion 61 and the inner straight edge portion 63, respectively. The leaflets 31 and 32 cooperate with one another and with the passageway 43 to provide a valve structure that can either close the passageway 43 or open the passageway 43 for passage of blood therethrough in one direction, as shown, for example, by the arrow 35 in FIG. 1.

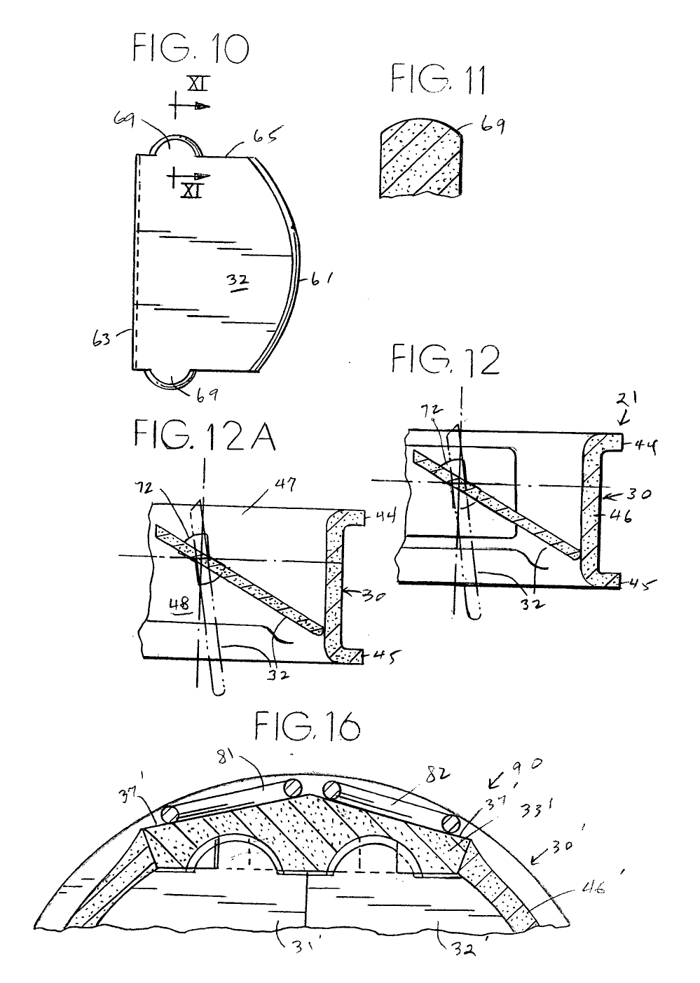

Each of the leaflets 31 and 32 has a perimeter configuration that is illustrated, for example, by the plan view of leaflet 32 in FIG. 10. Thus, each leaflet 31, 32 has a perimeter that includes an edge portion 61 that extends arcuately in a nearly circular configuration to define a leaflet outside edge region, and another edge portion 63 that extends straight to define an inside edge region. In the region of each leaflet 31 and 32 located between the end of each straight portion 63 and the adjacent end of each arcuate portion 61, a relatively short, straight end edge portion 65, 66 is defined on each leaflet 31 and 32, respectively. In each of the straight end edge portions 65, 66 of each leaflet 31 and 32, an integrally formed, flattened, approximately semicircular, preferably perimetrically rounded, outwardly-projecting, ear-like projection 69 is located. The straight edge portions 63 of each leaflet 31 and 32 is preferably beveled so as to permit these edge portions to abut one another in a complementary manner when the leaflets 31 and 32 are in a closed configuration across the passageway 43 of the prosthesis 21. The arcuate edge portions 61 are preferably provided with a rounded beveled region to permit each of these edge portions 61 to sealingly abut against adjacent regions of the medial side wall portions 46 when, as pivoting occurs, the edge portions abut thereagainst.

The flat interior face 48 of the integral bearing block 47 and also the flat interior face 55 of the separate bearing block 33 each has formed therein a pair of circumferentially (relative to the ring 30) spaced but adjacent cavities 71, 72, respectively. One cavity 71 in block 33 taken with a corresponding opposing cavity 71 in block 47 defines a pivot axis 75 (see FIG. 2) for a leaflet 31 while one cavity 72 in block 33 taken with an opposing cavity 72 in block 47 defines a pivot axis 76 for leaflet 32. Each ear 69 is configured to extend into a cavity 71 or 72 and to pivot relative thereto. During assembly of the prosthesis 21, with the circlip 34 removed from the grooves 57A and 75B (see FIG. 14), and with the block 33 loosened relative to the window 49, the ear-like projections 69 of the leaflet 31 are positioned each in a different cavity 71 of each block 47 and 33, and the ear-like projections 69 of the leaflet 32 are positioned each in a different cavity 72 of each block 47 and 33. Thereafter, the block 33 is seated in the window 49 and the circlip 34 is associated with the grooves 57A and 57B to complete prosthesis 21 assembly. Thus, assembly of prosthesis 21 is completed without flexing or distortion of any pyrolytic carbon component.

The interrelationship between the leaflets 31 and 32 and the cavities 71 and 72 in the prosthesis 21 is such that the leaflets 31 and 32 are each locatable (by pivoting) across a different portion of the passageway 43 with each ear-like projection 69 being pivotably associated with different one cavity 71 or 72, as the case may be, in each of the separate bearing block 33 and the integral bearing block region 47 and with the leaflets 31 and 32 being responsive to fluid pressure applied on an upstream side thereof (as indicated by the arrow 35) whereby the leaflets 31 and 32 functioning in combination are adapted to extend across and close the passageway 43 and thereby define a valve closed configuration. Leaflets 31 and 32 also pivot and open the passageway 43 and thereby define valve open configurations as illustrated, for example, in FIG. 12. The position of a leaflet 31 or 32 is a function of the fluid pressure applied thereagainst. The interrelationship between the leaflets 31 and 32, the ear-like projections 69, the blocks 33 and 47, and the cavities 71 and 72 in the assembled prosthesis 21 is such that the leaflets 31 and 32 cannot slip or change from their pivotable positions during operation of the prosthesis 21.

Each leaflet 31 and 32 may oscillate freely between fully closed and fully open positions (see, for example, FIG. 12), and each leaflet 31 and 32 pivots independently of the other about its respective eccentric hinging (or pivoting) axes 75, 76 from open to closed positions (inclusive). When viewed from a position along the pivot axis, each cavity 71 and 72 is preferably characterized by a butterfly-type of cavity perimeter configuration and each preferably has an internal spherical configuration that permits each associated ear-like projection 69 to have only limited pivotal movement over a predetermined number of degree angles. At each end of each maximum prechosen pivot, each associated ear-like projection 69 comes into abutment with an adjacent preferably vertically extending side wall portion 77 of the associated cavity 71 or 72, thereby to achieve a stop that achieves a restricted pivotal movement for each leaflet 31 and 32 across and within the passageway 43 of the ring member 30 in response to fluid pressure differentials developed in the cardiac system of a patient wherein the prosthesis 21 is implanted surgically with the direction of flow of blood being shown by the illustrative arrow 35. The indicated preferred configuration of the cavities 71 and 72 also serves to avoid potential stagnation of blood which might tend to otherwise occur in unused portions of the cavities 71 and 72 were they to be otherwise configured due to the preferred indicated restricted pivotal movement of the leaflets 31 and 32. The side portions 77 can be considered to co-act with the ear-like projections 69 to provide stop means for limiting pivotal movements of the leaflets 31 and 32.

In each leaflet 31 and 32, the ears 69 are disposed nearly diametrically opposite to each other (relative to the passageway 43). The respective dimensions between components are chosen so that a minute uniform clearance exists between spherical edge surfaces of each ear 69 and adjacent portions of associated cavities 71 and 72. Curved surface portions of each cavity 71 and 72 and of the associated projections 69 correspond preferably to complementary spherical segments. The cavities 71 and 72 cooperate with each other and with the associated ear like projections 69 to achieve self-aligning bearing assemblies for free and smooth pivotal movement of each leaflet 31 and 32 when in assembled association with the ring member 30.

A simple, effective structure is achieved by having one of the two bearing blocks, here block 47, be an integral part of the ring member 30 while the other bearing block, here block 33 is separate but readily and easily associated the window 49 defined in the side wall 46 of the annular or ring member 30. The preferred rectangular mating perimeter configurations of the block 33 and the window 49 enhances a firm and stable seating and sealing engagement between the bearing block 33 and the window 49.

The two pivot (hinging) axes 75 and 76 are each parallel to the diameter 40 of the ring 30, and each is spaced at a distance y (see FIG. 2) away from the diameter 40. The planes defined by the flat interior faces 55 and 48 are each perpendicular to the hinging axes 75, 76 and parallel to the edge portions 65, 66. The straight edge portions 65, 66 are preferably perpendicular to the respective hinging axes 75, 76, thereby to achieve free rotary movement of each leaflet 31 and 32. The ear-like projections 69 preferably have about the same thickness as that of the associated leaflet 31 or 32. Preferably, the outer peripheral arcuate edge portions 61 of the leaflets 31 and 32 each have a curved or rounded profile which is adapted to abut against the adjacent curvature of the inner surface of medial wall 46 at locations thereof where portions 61 rest when in the valve closed position, thereby to avoid jamming between the edges 61 and the wall 32, particularly due to possible fluidic back pressure that may occur against a leaflet 31 or 32.

Each leaflet 31 and 32 has a configuration that covers about half of a selected area across the passageway 43 through the ring member 30. The straight perimeter edge portions 63 and 64 of the leaflet 31 and 32 are preferably beveled and these edge portions 63 and 64 preferably (as shown) meet together when the leaflets 31 and 32 are in the valve closed configuration, and in this closed configuration each leaflet 31 and 32 is somewhat inclined relative to the other whereby fluid pressure against the faces of the leaflets 31 and 32 urges them into the closed configuration. In this valve closed configuration, the arcuate edge portions 61 and 62 preferably abut and engage (as shown) against the inside of the adjacent portions of the medial side wall portions 46 of the ring member 30 while the respective straight edge portions 65 and 66 are located adjacent to portions of each of respective the flat faces 48 and 54. In the closed configuration, the leaflets 31 and 32 are preferably configured to provide about their periphery a seal across the passageway 43 that prevents the flow of blood therethrough. Preferably a leakproof joint is achieved that prevents back flow of blood when the leaflets 31 and 32 are in their closed positions.

The flat chord-like (when the ring 30 is viewed axially) edges 65, 66 of the leaflets 31 and 32 as well as to the respective hinging or pivoting axes 75, 76 function to wipe and to clean blood on the flat faces 48 and 55 of the bearing housings 47 and 33 during each oscillation of the leaflets 31 and 32. The preferably spherically curved peripheral edge of each ear-like projection 69 functions to sweep the adjacent spherical surfaces of each cavity or recess 71 and 72.

Various methods of the prior art can be used in fabricating pyrolytic carbon containing components employed in the present invention. Typically, a component with a carbon surface is heated to beyond 1,000° C. to achieve a hard and naturally polished surface. Higher temperatures give greater hardness depth relative to the surface. See, for example, Bokros U.S. Pat. No. 3,676,179.

For example, in one process, carbon black powder is pressed under high pressure to make rods or other shapes. The shapes are machined and articles (components) are produced. In the present situation, the leaflets and the ring member are produced. These articles are then heated in a controlled atmosphere to 1,200° C. or above, the temperature selected being influenced by the desired structure. Thus, the resulting pyrolytic carbon components of an inventive prosthesis embodiment, such as the leaflets, for example, are characteristically heat treated and hardened but not coated using conventional technology. Typically, the pyrolytic carbon components are inert and light in weight and density.

To achieve a precisely dimensioned product, one may position a preformed but unfinished (or “blank”) separate bearing block in the bearing block window of a preformed but unfinished (or “blank”) annular ring structure and retain the unfinished bearing block in position with the circlip. Surface portions of this combination of ring structure and separate bearing block can then be machined and the bearing recesses or cavities formed therein. Thereafter, the circlip can be removed, the separate bearing block loosened from the window, the leaflet ear-like projections duly positioned in the recesses, and the separate bearing block reseated in the window and retained by the circlip to complete prosthesis assembly. The steps of this method of prosthesis preparation can be, for example, characterized as follows:

(a) forming in a blank for the annular structure the bearing block receiving window including window perimeter portions;

(b) forming perimeter portions about a blank for the separate bearing block, these perimeter portions being cooperatively engageable with the window perimeter portions whereby the blank for the separate bearing block is seatable within the bearing block receiving window;

(c) forming groove means in the blank for the annular structure, the groove means being adjacent to the bearing block receiving window, and associating circlip means with the groove means, whereby said blanks are associated together in a combination;

(d) machining surface regions of said combination whereby
a flat interior face is defined upon said blank for said separate bearing block,
said integral bearing block is defined in said blank for said annular structure with a flat interior face for said integral bearing block that is in spaced, parallel relationship to said flat interior face of said separate bearing block,
each of said flat interior faces is parallel to a longitudinal axis defined in said annular structure; and
each of said flat interior faces has defined therein said pair of spaced bearing recesses, thereby to form said annular structure and said separate bearing block;

(e) said circlip means is dissociated from said groove means and said separate bearing block is separated from said annular structure while said ear-like projections of each of said leaflets are pivotably each engaged with one said bearing recess in each said bearing blocks; and

(f) said separate bearing block is reengaged with said window and said circlip means is reassociated with said groove means, thereby to fabricate said prosthetic heart valve.

Various other and further embodiment applications, structures and the like will be apparent to those skilled in the art from the teachings herein provided and no undue limitations are to be drawn therefrom.

1. A prosthetic heart valve assembly comprising in combination:
(a) an annular structure having a longitudinal axis, generally cylindrical medial sidewall portions defining a central passageway therethrough, and outwardly flanged terminal rim portions, said side wall portions defining an integral bearing block and a generally diametrically opposed bearing block receiving window;
(b) a separate bearing block for seating in said bearing block receiving window;
(c) said separate bearing block and said bearing block receiving window having cooperating respective perimeter portions whereby said separate bearing block is received within and sealingly engaged within said bearing block receiving window;
(d) said integral bearing block and said separate bearing block each having a flat interior face that has defined therein a pair of circumferentially spaced bearing recesses, said interior faces being parallel to each other and to said longitudinal axis;
(e) groove means defined in said side wall portions adjacent to said bearing block receiving window and circlip means demountably associatable with said groove means for maintaining said separate bearing block seated in said bearing block receiving window;
(f) a pair of leaflets, each one disposed across said passageway, each said leaflet having a generally flattened body portion defined by a perimeter that includes a generally arcuately extending outside edge region, a straight inside edge region, and a pair of outwardly extending, integral, peripherally rounded, ear-like projections, each said ear-like projection being located between a different adjacent pair of said outside edge region and said inside edge region; and
(g) the interrelationship between said leaflets, said annular structure, and said bearing blocks being such that said leaflets are each locatable across a different but adjacent portion of said passageway with each said ear-like projection being pivotably associated with different one recess in each of said bearing blocks and with said leaflets being pivotable responsive to fluid pressure applied on an upstream side of said passageway whereby said leaflets in combination are adapted to extend across and close said passageway and thereby define a valve closed configuration and also to pivot and open said passageway and thereby define valve open configuration.

2. The prosthetic heart valve assembly of claim 1 wherein said annular structure, said separate bearing block, and said leaflets each have surface portions comprised of pyrolytic carbon.

3. The prosthetic heart valve assembly of claim 1 wherein said circlip is comprised of non-rusting spring steel.

4. The prosthetic heart valve assembly of claim 1 wherein, when said leaflets are in said valve closed configuration, said straight inside edge portion of each said leaflet is in adjacent, contacting relationship with the other, and said arcuately extending outside edge region of each said leaflet is in adjacent contacting relationship with a different interior side wall portion of said annular structure whereby said passageway is substantially closed.

5. The prosthetic heart valve assembly of claim 1 wherein the extent of pivotal opening and closing movement of each said leaflet is limited by the internal configuration of each said recess and wherein each said recess has internal wall-like portions against which facial portions of the associated said ear-like projection abut at the limit of closing and of opening pivotal movement of said ear-like projection.

6. The prosthetic heart valve of claim 1 wherein said separate bearing block and said bearing block receiving window are associated with locating pin means which cooperatively associate with one another to position and locate said separate bearing block relative to said receiving window.

7. A prosthetic heart valve assembly comprising in combination:
(a) an annular structure having a longitudinal axis, generally cylindrical medial sidewall portions defining a central passageway therethrough, and outwardly flanged terminal rim portions, said side wall portions defining at one location thereof an integral, radially thickened bearing block portion having a flat interior face that extends across said portion of said passageway and that extends parallel relative to said longitudinal axis, and said side wall portions also defining at a generally diametrically opposed location relative to said integral bearing block portion a bearing block receiving window having window perimeter edge portions that are radially inclined;
(b) a separate bearing block for seating in said bearing block receiving window, said separate bearing block having radially inclined block perimeter edge portions that are configured to matingly engage with and seat against said window edge perimeter portions when said bearing block is inserted into said bearing block receiving window from an exterior location relative to said annular structure, said separate bearing block having a flat interior face that extends across said portion of said passageway, perpendicular to said longitudinal axis, and also parallel to said flat interior face of said integral bearing block portion when said separate bearing block is so seated in said bearing block receiving window;
(c) said window perimeter portions further having groove means defined therein located adjacent to said separate bearing block perimeter edge portions when said separate bearing block is so seated against said window edge perimeter portions, and
(d) a circular circlip for positioning in said groove and for retaining said separate bearing block in association with said annular structure when said separate bearing block is so seated in said bearing block receiving window;
(e) a pair of leaflets, each one disposed across said passageway, each said leaflet having a generally flattened body portion defined by a perimeter that includes a generally arcuately extending outside edge region, a straight inside edge region, and a pair of outwardly extending, integral, peripherally rounded, ear-like projections, each said projection being located between a different adjacent pair of said outside edge region and said inside edge region;
(f) said flat interior face of each of said integral bearing block portion and said separately formed bearing block having defined therein a pair of circumferentially spaced but adjacent cavities; and
(g) the interrelationship between said leaflets and said cavities in said assembly being such that said leaflets are each locatable across a different portion of said passageway with each said ear-like projection being pivotably associated with different one cavity in each of said separate bearing block and said integral bearing block portion and with said leaflets being responsive to fluid pressure applied on an upstream side thereof whereby said leaflets in combination are adapted to extend across and close said passageway and thereby define a valve closed configuration and also to pivot and open said passageway and thereby define valve open configurations.

8. The prosthetic heart valve assembly of claim 7 wherein surface portions of said annular structure, said separate bearing block and said leaflets are each comprised of pyrolytic carbon.

9. The prosthetic heart valve assembly of claim 8 wherein said circlip is cross-sectionally circular.

10. The prosthetic heart valve assembly of claim 8 wherein said circlip is comprised of non-rusting spring steel.

11. The prosthetic heart valve assembly of claim 7 wherein, when said leaflets are in said valve closed configuration, said straight inside edge portion of each said leaflet is in adjacent, contacting relationship with the other, and said arcuately extending outside edge region of each said leaflet is in adjacent contacting relationship with a different interior side wall portion of said annular structure whereby said passageway is substantially closed.

12. The prosthetic heart valve assembly of claim 11 wherein, in said valve closed configuration, said straight inside edge portion of each said leaflet is configured to seat against the other.

13. The prosthetic heart valve assembly of claim 11 wherein, in said valve closed configuration, said arcuately extending outside edge region of each said leaflet is configured to seat against adjacent side wall portions of said annular structure.

14. The prosthetic heart valve assembly of claim 7 wherein peripheral edge portions of each of said ear like projections and interior surface portions of each of said cavities each have spherical curvatures and wherein adjacent edge portions of each of said ear like projections is matingly engageable with the respective associated one of said cavities whereby each said ear-like projection is freely pivotable relative to the associated said cavity.

15. The prosthetic heart valve assembly of claim 14 wherein the extent of pivotal opening and closing movement of each said leaflet is limited by the internal configuration of each said cavity.

16. The prosthetic heart valve assembly of claim 14 wherein each cavity has internal wall-like portions against which facial portions of the associated said ear-like projection abut at the limit of closing and of opening pivotal movement of said ear-like projection.

17. The prosthetic heart valve assembly of claim 7 wherein said separate bearing block and said bearing block receiving window each have a generally rectangular perimeter configuration.

18. The prosthetic heart valve assembly of claim 7 wherein in regions thereof adjacent to said bearing block receiving window said annular structure is radially thickened.

19. The prosthetic heart valve assembly of claim 7 wherein said separate bearing block and said bearing block receiving window are associated with locating pin means which cooperatively associate with one another to position and locate said separate bearing block relative to said receiving window.

20. A method for fabricating the prosthetic heart valve assembly of claim 1 comprising the steps of:
(a) forming in a blank for said annular structure said bearing block receiving window with window perimeter portions;
(b) forming about a blank for said separate bearing block perimeter portions that are cooperatively engageable with said window perimeter portions whereby said blank for said separate bearing block is seatable within said bearing block receiving window;
(c) forming groove means in said blank for said annular structure that are adjacent to said bearing block receiving window and associating circlip means with said groove means, whereby said blanks are associated together in a combination;
(d) machining surface regions of said combination whereby;
a flat interior face is defined upon said blank for said separate bearing block,
said integral bearing block is defined in said blank for said annular structure with a flat interior face for said integral bearing block that is in spaced, parallel relationship to said flat interior face of said separate bearing block,
each of said flat interior faces is parallel to a longitudinal axis defined in said annular structure; and
each of said flat interior faces has defined therein said pair of spaced bearing recesses, thereby to form said annular structure and said separate bearing block;
(e) said circlip means is dissociated from said groove means and said separate bearing block is separated from said annular structure while said ear-like projections of each of said leaflets are pivotably each engaged with one said bearing recess in each said bearing blocks; and
(f) said separate bearing block is reengaged with said window and said circlip means is reassociated with said groove means, thereby to fabricate said prosthetic heart valve.
